(19) United States
(12) Patent Application Publication
(10) Pub. No.: US 2007/0141180 A1
(43) Pub. Date: Jun. 21, 2007

(54) Succulent plant water

(76) Inventors: Jong II Shin, McAllen, TX (US); William L. Davis, McAllen, TX (US)

Correspondence Address: William F. Ryann, Ste. 1629, 115 E. Travis St., San Antonio, TX, 78205, US

(21) Appl. No.: 11/352,577

(22) Filed: Feb. 13, 2006

Related U.S. Application Data
(63) Continuation-in-part of application No. 11/304,856, filed on Dec. 15, 2005.

(51) Int. Cl. A61K 36/896; A61K 36/886; A61K 36/254; A61K 36/42; A61K 36/28; A61K 36/33

(52) U.S. Cl. 424/728; 424/753; 424/767; 424/758; 424/744; 424/764

(57) ABSTRACT

Water is obtained from a succulent plant. This may be achieved by denaturing a water-based sap of the succulent plant from a water-storing portion of the succulent plant. Water may then be evaporated from the resulting liquefied sap of the succulent plant. As many succulent plants thrive in arid climates, methods described herein provide a mechanism by which substantial quantities of pure drinking water may be obtained in a large scale cost-effective manner.

### PRIORITY CLAIM

This Patent Document is a Continuation-In-Part of Ser. No. 11/304,856, Nopal Extract (Jong I. Shin).

### BACKGROUND

Embodiments described relate to a water obtained from a succulent plant. In particular, embodiments relate to obtaining water from a water-based sap within a water-storing portion of a succulent plant.

### BACKGROUND OF THE RELATED ART

Water is a required and essential part of every day life. Often taken for granted, the availability of fresh clean water for human consumption is of ever increasing importance as society evolves and the world's population continues to grow at an exponential rate. Nevertheless, the issue of water scarcity has historically been of concern in survivalist situations. For example, people at sea, hiking in the wilderness, or residing in highly arid climates may employ a variety of available tools for obtaining drinking water from seemingly non-conventional sources. These effective, but often inefficient, tools may include the desalination of saltwater, extraction of fresh water from fish, or, as described further below, obtaining water from plants by taking advantage of the natural process of transpiration.

Transpiration is the process by which plants naturally transport water toward their leaves or needles and eventually into the air. For example, the transpiration of water as a constituent of plant sap may naturally proceed through a tree toward its branches and leaves, with the water eventually being released from the leaves of the tree. Certain trees transpire water in this manner at particularly regular or predictable rates. Varieties of oak, pine, walnut and certain fruit trees may be especially good candidates for a collection of fresh water by taking advantage of the transpiration process as described further below.

As water escapes from tree leaves in the process of transpiration, it may be captured. For example, a clear plastic bag may be tied off around a tree branch whereby water exiting the tree through its leaves can evaporate and collect at the inner surface of the bag, ultimately aggregating as water at the bottom of the bag.

This method of collecting water from a tree includes inherent purity advantages over collection of water from more conventional sources such as groundwater, streams, reservoirs and the like. Unlike more conventional water sources, once water is stored within the tree it is naturally isolated from the potential contaminants of the outside world. Furthermore, as the water transpires within the tree, it is filtered by the superior and natural filtering mechanism of the tree itself. Thus, substantially pure water is available for collection from certain plants and trees via transpiration. On the other hand, water collected from an insecure environment such as groundwater invariably includes contaminants due to the naturally exposed nature of the water source.

Unfortunately, current water collection methods through natural transpiration of water from a tree are extremely inefficient. That is, while potentially effective for a hiker with a clear plastic bag, it is not practical to collect water via this mechanism on a large scale. This is because the volume of water obtainable from a tree is limited to the amount of water transpired and released from its leaves over a given period. Even from a large tree, the practical amount of water obtainable through leaf transpiration is limited to a few quarts per day. Once more, efforts to increase the volume of water collected, for example, by maximizing the number of bagged branches, have deleterious effects on the tree. Thus, for the price of a few gallons of water drawn from a tree, for example, during a given growing season, the tree is likely killed or substantially impaired and no longer available as a useful source of water for human consumption.

In addition to the noted inefficiencies of leaf transpiration, plants which transpire water to any noticeable degree, like those indicated above, are generally found in naturally moderate climates. More severe arid or desert climates are unlikely to have an abundance of plants which transpire significant quantities of water vapor directly into the air. Thus, in areas where water is scarcest, the option of water collection by taking advantage of the transpiration process may simply be implausible.

Unlike most trees, succulent plants, such as cacti, aloe, and others have an extremely high water-storing capacity. Furthermore, most succulent plants exhibit even more effective water purification capacity than that witnessed in trees as described above. Succulent plants, as described here, are those having a water-storing portion made up of mostly water. As an example, the nopal plant is a succulent plant which may be any of various cacti of the genera Nopalea or Opuntia, including the prickly pear (Opuntia Ficus-Indica) and similar species having a vegetable, fleshy, oval pad that is over 90% water.

Unfortunately, succulent plants such as the nopal are adept not only at water-storing, but also at water retention given their natural habitat of more arid or desert climates as alluded to above. Succulent plants exhibit only a negligible amount of water naturally transpiring or released from their water-storing portions directly into the air. Furthermore, even the water which is retained is mostly trapped within the water-based sap of the water-storing portion along with other natural substances. Thus, the succulent plant remains ineffective as a fresh water source for human consumption. In fact, in the case of the nopal plant, its sap has been historically employed as a construction material which hardens to exhibit properties similar to a dried glue.

### SUMMARY

Embodiments herein describe water that is obtained from a water-storing portion of a succulent plant. The water-storing portion of the succulent plant includes a water-based sap which is isolated from the water-storing portion. The water is then drawn from the water-based sap.

### BRIEF DESCRIPTION OF THE DRAWINGS

FIG. 1 is an overview of an embodiment of a succulent plant processing mechanism (SPPM).

FIG. 2 is a side cross sectional view of a nopal pad on the SPPM, taken from section lines 2-2 of FIG. 1.

FIG. 3 is an overview of an embodiment of a succulent plant water extract processing system (SPWEPS).

FIG. 4 FIG. 4 is a perspective view of an embodiment of a bottle filler for coupling to the SPWEPS of FIG. 3.

FIG. 5 is a flow chart summarizing an embodiment of extracting water from a water-storing portion of a succulent plant.

### DETAILED DESCRIPTION

Embodiments are described with reference to certain methods of obtaining water from succulent plants. These may include particular embodiments of denaturing a water-based sap with enzymes to provide a workable liquefied sap from which water may be evaporated. Regardless, embodiments described herein include the removal of the water-based sap from a water-storing portion of the succulent plant.

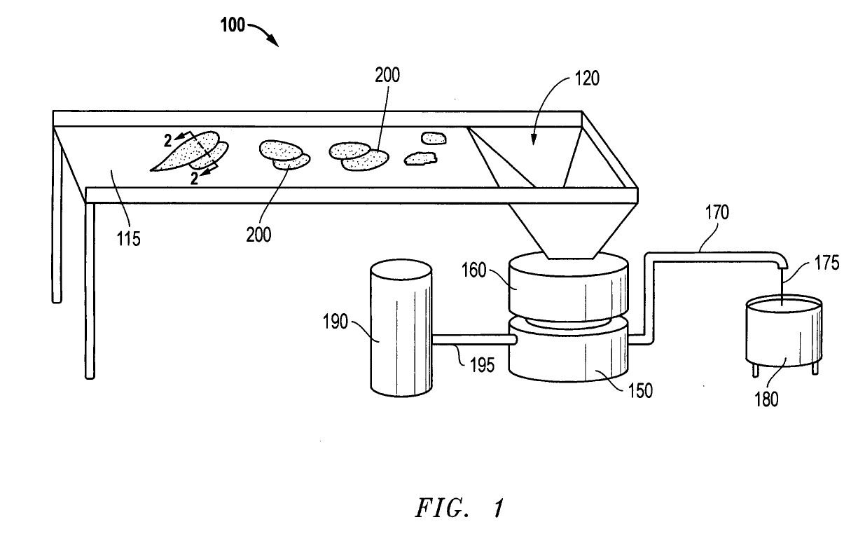

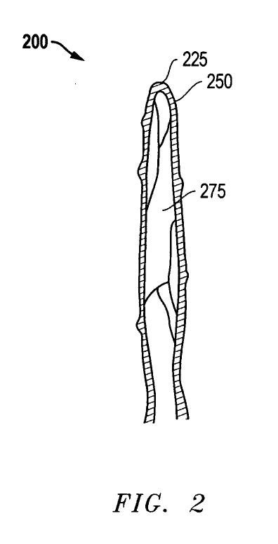

With reference to FIGS. 1 and 2, a water-storing portion of a succulent plant is shown in the form of a nopal pad 200. Succulent plants, as described here, are those having a water-storing portion made up of mostly water. In the case of the nopal pad 200, it is itself a water-storing portion that is over 90% water. The nopal is of the Nopalea or Opuntia, including the common prickly pear cactus (Opuntia Ficus-Indica). However, in alternate embodiments, water may be obtained from a variety of water-storing portions of succulent plants. These may include plants whose water-storing portion requires the addition of enzymes or other denaturing constituents in order to facilitate the release of water therefrom. Embodiments of succulent plants may include aloe, yucca plants, agaves, and a variety of traditional cacti from barrel cacti to organ pipes. Indeed, other varieties of plants may be viewed as succulents to the extent that they include a water-storing portion that is mostly water, such as certain fruiting plants (e.g. citrus fruits and various melons).

With the nopal pad 200 as an example of a water-storing portion of a succulent plant, a manner of obtaining water therefrom is described further herein. The nopal pads 200 are initially harvested and washed with water and a conventional food grade sanitizer. Then, with specific reference to the embodiment of FIG. 1, the pads 200 are then placed at a conveyor belt 115 of a succulent plant processing mechanism (SPPM) 100. The conveyor belt 115 advances the nopal pads 200 to a receptacle 120 which directs the nopal pads 200 to a conventional grinder box 160 where the spines, skins, and other solid pieces of the nopal pads 200 are removed.

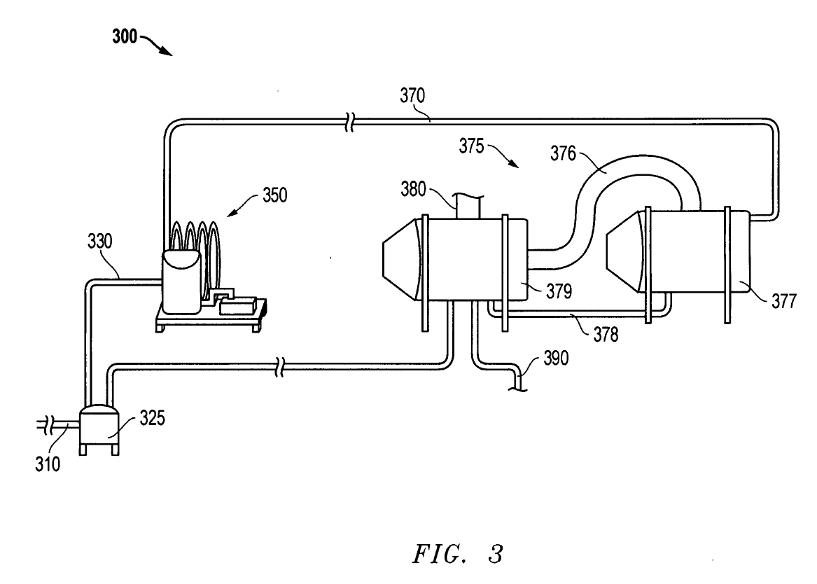

Once the nopal pads 200 have been ground as described, a water-based nopal sap 175 may be separated from the remaining solid fibrous waste of the nopal pads 200. Cold compression may be employed to help achieve this separation (e.g. see the cold compression chamber 150). With the aid of enzyme activity noted below, the nopal sap 175 may then be drawn through an outlet pipe 170 and emptied into a receiving tank 180. Initially the nopal sap 170 is of a gel-like consistency. Therefore, in order to denature and more fully liquefy the nopal sap 175, enzymes may be added to the compression chamber 150 before advancing the nopal sap 175 to the receiving tank 180 shown. The enzymes may be of a cellulase variety delivered by way of an enzyme line 195 as shown (coming from an enzyme tank 190). The amount of enzymes added to the nopal sap 175 is negligible in volume. Nevertheless, the enzymes may be a necessary constituent of the obtained nopal sap 175 in order to provide a workable liquid from which to extract water as described further herein. That is, in order to effectively push the nopal sap 175 through the succulent plant water extract processing system (SPWEPS) 300 and obtain water therefrom, the nopal sap 175 may need to actually be a liquefied nopal sap 175 exhibiting properties similar to water itself (see FIG. 3).

With brief added reference to FIG. 3, the liquefied nopal sap 175 may now also be run through filtration, pasteurization, and any other desired processing before placement in nopal sap tanks for delivery to the SPWEPS 300. For example, in one embodiment, a 1 micron to 10 micron filtration may be employed. The liquefied nopal sap 175 may now be processed by the SPWEPS 300 as described further below.

Referring now to FIG. 2, with additional reference to FIG. 1, a side cross sectional view of a nopal pad 200 is shown. While the nopal pad 200 is over 90% water, this water is locked within the nopal sap 175 and initially unavailable for processing by conventional means. Thus, the nopal, like other succulent plants is particularly adept at preserving its water in light of the arid climates in which it is likely to be found.

Most of the water of the nopal pad 200 is found at its cortex 275 where a cellular matrix of mucilage which encapsulates the water. Encapsulating the water in this manner helps avoid transpiration and loss of water vapor from the nopal pad 200. The mucilage, water and various other ingredients make up the initially gluey gel-like nopal sap 175 adept at retaining its water.

In addition to the inherent water retaining properties of the mucilage and nopal sap 175, the nopal pad 200 itself is constructed and operates in a manner so as to maximize water collection and retention. For example, the nopal pad 200 includes a waxy epidermis 225 which can be a barrier to the escape of moisture. Additionally, the stomata within the cuticle 250 of the nopal pad 250, through which CO₂ is received from the surrounding atmosphere, are configured to avoid air currents and other factors that might encourage the release of water vapor. Furthermore, the nopal is most active for exchange of air and absorption of water and minerals at night with the sun unable to have an affect on the water content of the plant. This provides a further water collection advantage to the nopal, given that much of the water available in arid or desert climates comes in the form of evening dew and mist. All in all, these factors make the nopal and many other succulents extremely effective water collectors; so much so, that in spite of being a part of a desert plant, the nopal pad 200 is made up of over 90% water.

In addition to water collection, succulents such as the nopal often provide excellent reservoirs of substantially pure water. That is, aside from the natural constituents of the cortex 275, there are likely no other measurable ingredients or contaminants to be found at the interior of the nopal pad 200. Unlike many plants, a succulent such as the nopal is likely to take in a significant percentage of its water from the collection and absorption of mist and dew as noted above. Whereas ground or surface water is susceptible to ground contaminants such as sewage, waste, chemical spills and the like, mist and dew form in the air and are much less likely contaminated. As a result, a significant portion of the water taken in by the nopal comes in a substantially pure form. Additionally, once absorbed, the water is naturally filtered and retained by a cellular matrix of mucilage as noted above which acts to prevent the absorption of contaminants. Thus, succulents such as the nopal are effective high volume collectors of substantially pure water.

In spite of the impressively high volume of substantially pure water contained at the water-storing portion, many succulents such as the nopal do not readily yield their water to the thirsty passer by. That is, as noted above, the water of the nopal pad 200 is not obtained merely by squeezing the pad 200. Rather, in the embodiment described, enzymes are necessary to liquefy the nopal sap 175 to a workable state. The enzymes added are of a cellulase variety and, in the embodiment described, make the separation of the water from the remainder of the nopal sap 175 possible. This separation and collection of substantially pure water for human consumption is described herein below.

Referring now to FIG. 3 with added reference to FIG. 1, a liquefied nopal sap 175 is delivered to a succulent plant water extract processing system (SPWEPS) 300 via a delivery line 310. The liquefied nopal sap 175 is initially advanced further by a balance tank 325. From here it is eventually directed to an evaporator 375 for removal of water therefrom, and then packaged for human consumption.

The balance tank 325 itself may be employed as a safety measure to ensure a constant flow of liquid to the evaporator 375 while the evaporator 375 is running. That is, if a supply of liquefied nopal sap 175 is unavailable, the balance tank 325 may pull previously stored water from a nearby source (not shown) for advancing through the running evaporator 375. In this manner, damage to the evaporator 375 is avoided.

In addition to the balance tank 325, a pasteurizer 350 may be disposed between the balance tank 325 and the evaporator 375. Further, while, the SPWEPS 300 is shown with the balance tank 325 preceding the pasteurizer 350, followed by the evaporator 375 and perhaps a fermentation tank assembly as described below, the liquefied nopal sap 175 may take a variety of application routes. For example, in one embodiment, the liquefied nopal sap 175 may bypass by the pasteurizer 350 without pasteurization and proceed directly to the evaporator 375 for removal of water therefrom. As the evaporated water should contain no organic matter, the need for pasteurization may not exist. On the other hand, in an embodiment where the liquefied nopal sap 175 is to provide a concentrated organic extract in addition to a source of water, pasteurization may be desirable at some point in the process. A variety of processing options are possible. Additionally, sample ports may be dispersed throughout the lines (120, 130, 170, 178, 190, 193) for testing contamination or taking other readings to help establish the particular application route for the liquefied nopal sap 175 through the SPWEPS 300.

As indicated above, the evaporator 375 may be employed to withdraw the water from the liquefied nopal sap 175. That is, upon delivery to the evaporator 375, the liquefied nopal sap 175 includes dietary ingredients and constituents such as certain vitamins, minerals, and amino acids. However, the vast majority of the liquefied nopal sap 175 at this point is water (i.e. perhaps about 95%). Therefore, the evaporator 375 may be employed to remove this water so that it may be packaged for human consumption.

The evaporator 375 shown in FIG. 3, includes first 377 and second 379 evaporator tanks. However, the evaporator 375 may include any number of evaporator tanks. For example, in one embodiment, the evaporator 375 includes 4 tanks. In this embodiment, the first evaporator tank 377 with liquefied nopal sap 175 therein may be heated such that water vapor rises from the liquefied nopal sap 175 and is directed to a vapor tube 376 leaving an initial concentrate of liquefied nopal sap 175. This initial concentrate of liquefied nopal sap 175 may then be advanced via a tank line 378 to the second evaporator tank 379 for further evaporation of water therefrom. Following evaporation in the second tank 379, the liquefied nopal sap 175 may similarly be advanced to additional evaporative tanks, such as the third and fourth of the embodiment described here. This process may continue until the efficient and practical maximum of recoverable water has been obtained from the liquefied nopal sap 175.

The water obtained may additionally be filtered to ensure a palatable taste, for example by employing an in-line activated charcoal filter. UV treatment may similarly be employed as a precautionary measure, given that the water is for human consumption.

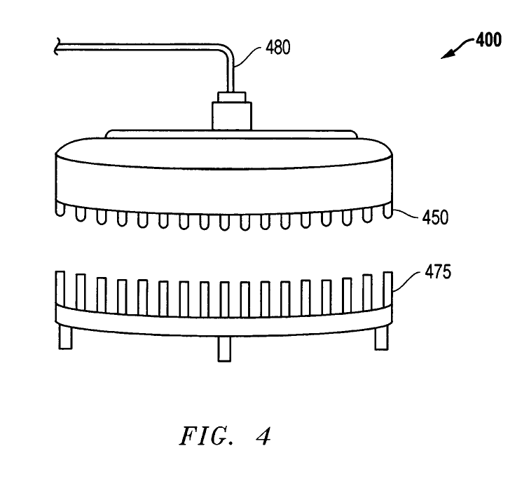

Referring now to FIG. 4, a bottle filler 400 is shown. A naturally pure supply of water for human consumption may be supplied to the bottle filler via bottle line 480. The bottle filler 400 is shown with valves 450 coupled to the bottle line 480 and adjacently above bottle supports 475. In the embodiment shown, the bottle filler 400 is rotable and may be positioned adjacent to a conveyor belt for delivering sorted empty bottles. In one embodiment the bottle filler 400 may, while rotating, sequentially secure bottles between the valves 450 and bottle supports 475, and fill the bottles. The bottle filler 400 may then release filled bottles of water for capping, casing and any further packaging. Thus, pure water derived from the water-storing portion of a succulent plant may be available for shipping and ultimate consumer consumption.

This water has been derived from a succulent plant in a manner that takes advantage of the natural water volume and filtering capacity of succulent plants. Additionally, in arid climates, where water sources are generally scarce but succulent plants may be abundantly cultivated, transportation costs associated with the acquisition of substantial quantities of pure drinking water can be virtually eliminated.

In addition to being an excellent source of water for human consumption, liquefied nopal sap 175 naturally contains a variety of dietary ingredients including vitamins, minerals, amino acids and a host of other phytochemicals. Therefore, embodiments of liquefied nopal sap 175 may also be concentrated anywhere from about 3:1 to about 65:1 or more. This may be achieved by the evaporator 375 with removal of the water, for example, to at least about a 3:1 ratio of water removed to the remaining concentrate thereof. That is, liquefied nopal sap 175 may be split into the components of purified water and a nopal extract concentrate in liquid or solid form.

In light of the possibility of also forming an organic nopal extract concentrate, the liquefied nopal sap 175 may be run through the pasteurizer 350 and pasteurized by conventional means. Additionally, the evaporator 375 may also then lead to a fermentation tank assembly (not shown) for the application of food process fermentation to the pasteurized nopal extract concentrate. Fermented and concentrated nopal extract may then be bottled as a drinkable health supplement for consumer use.

Whether a water, or a concentrated extract, products described herein are for human consumption. As a result, additional ingredients may be added to the prior to final bottling or packaging. These ingredients may include polyunsaturated fatty acids, chromium, momordica charantia, ginseng, ocimum sanctum, gymnema sylvestre, and fibers including trigonella foenum graecum and linum usitatissimum (i.e. flaxseed).

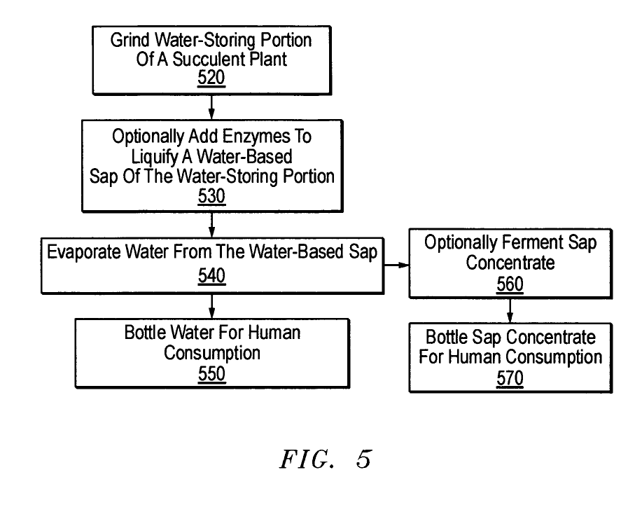

Referring now to the flow-chart of FIG. 5, and with additional reference to FIGS. 1-4, a method of obtaining water for human consumption from the water-storing portion of a succulent plant is described. As noted above, the water-storing portion of the succulent plant is ground (see 520). In the case of the nopal, its water-storing pad is removed and processed through an SPPM 100, where a conventional grinder box 160 is employed to separate the spines and skins of the nopal pads 200 from a water-based nopal sap 175. As indicated at 530, enzymes are then optionally added to denature the water-based sap of the water-storing portion, for example, where the water-based sap is naturally unworkable. This is the case with respect to the water-based nopal sap 175 for which cold-compression is also employed in separating out the sap.

With a liquefied water-based sap available, water may be evaporated therefrom as indicated at 540. This water may then be bottled for human consumption (see 550). In conjunction with the production of the water via evaporation, a beneficial concentrate of the sap may also be formed. The sap concentrate may also be collected and bottled for human consumption (see 560, 570).

Embodiments above describe efficient methods of obtaining water from succulent plants. These methods may be practically employed on a large scale due to the high volume of water obtainable from a succulent plant. Once more, the water-storing portion of a succulent plant may be repeatedly replaceable by the plant without deleterious effects on the plant, as is the case of the nopal pad of the succulent nopal plant. Furthermore, even though succulent plants may not readily release water, embodiments above describe methods of obtaining a substantially pure water from the succulent plants. Therefore, these embodiments describe methods for obtaining water from succulent plants by methods that take advantage of the natural water volume and filtering capacity of succulent plants. Additionally, in arid climates, where water sources are generally scarce but succulent plants may be abundantly cultivated, transportation costs associated with the acquisition of substantial quantities of pure drinking water may be virtually eliminated by use of the methods described herein.

We claim:

1. Water obtained from a water-storing portion of a succulent plant, the water-storing portion containing a water-based sap liquefied with an enzyme for extracting of said water therefrom.

2. The water of claim 1 wherein the enzyme is a cellulase enzyme.

3. The water of claim 1, wherein the succulent plant is one of aloe, yucca, agaves, a fruiting plant, and a traditional cactus.

4. The water of claim 1, wherein one of a polyunsaturated fatty acid, chromium, momordica charantia, ginseng, ocimum sanctum, gymnema sylvestre, and a fiber is added thereto.

5. The water of claim 1 wherein the water-storing portion is comprised of more than about 90% of said water.

6. The water of claim 5 wherein the water-storing portion is a nopal pad.

7. Water obtained from a water-storing portion of a succulent plant, the water-storing portion containing a water-based sap, the water-based sap provided for extracting said water therefrom for human consumption.

8. The water of claim 7 wherein the water-based sap is in a liquefied form from application of a cellulase enzyme thereto to allow the extracting by evaporation.

9. A method of acquiring water for human consumption comprising:
obtaining a water-based sap from a succulent plant; and
extracting the water from the water-based sap.

10. The method of claim 9 wherein said obtaining further comprises:
mechanically grinding a water-storing portion of the succulent plant; and
separating solid pieces of the water-storing portion from the water-based sap.

11. The method of claim 10 wherein said obtaining further comprises applying cold compression.

12. The method of claim 9 further comprising denaturing the water-based sap into a more liquified state in conjunction with said obtaining.

13. The method of claim 12 wherein said denaturing comprises adding a cellulase enzyme to the water-based sap.

14. The method of claim 9 wherein said extracting comprises evaporating the water from the water-based sap.

15. The method of claim 14 further comprising collecting a concentrate of water-based sap for human consumption.

16. The method of claim 15 further comprising pasteurizing the water-based sap before said collecting.

17. The method of claim 15 further comprising fermenting the concentrate of the water-based sap.

18. The method of claim 15 further comprising adding one of a polyunsaturated fatty acid, chromium, momordica charantia, ginseng, ocimum sanctum, gymnema sylvestre, and a fiber to the concentrate.

19. The method of claim 15 wherein said evaporating removes water from the water-based sap to at least about a 3:1 ratio of water removed to the remaining concentrate thereof.
